(19) United States
(12) Patent Application Publication
(10) Pub. No.: US 2007/0244515 A1
(43) Pub. Date: Oct. 18, 2007

(54) Multipurpose Surgical Tool

(76) Inventor: Medhat Youssef Zakher Fanous, Bloomfield, NJ (US)

Correspondence Address: ST. ONGE STEWARD JOHNSTON & REENS, LLC, 986 BEDFORD STREET, STAMFORD, CT, 06905-5619, US

(21) Appl. No.: 11/735,183

(22) Filed: Apr. 13, 2007

Related U.S. Application Data
(63) Continuation of application No. PCT/NZ05/00263, filed on Oct. 13, 2005.

(30) Foreign Application Priority Data
Oct. 14, 2004 (NZ) 535958

(51) Int. Cl. A61B 17/00; A61B 17/32

(52) U.S. Cl. 606/205; 606/174

(57) ABSTRACT

The invention relates to a surgical tool. In particular, the invention relates to a multi-functional surgical tool that has a cutting means, and a grasping means, with a buffer region between the cutting and grasping means. The invention particularly relates to a multi-functional surgical tool that is suitable for use in endoscopic or laparoscopic surgery, but may also be used in open surgery.

### CROSS-REFERENCE TO RELATED APPLICATIONS

The present application is a continuation of pending International patent application PCT/NZ2005/000263 filed on Oct. 13, 2005 which designates the United States and claims priority from New Zealand patent application 535958 filed on Oct. 14, 2004, the content of which is incorporated herein by reference.

### FIELD OF THE INVENTION

This invention relates to a surgical tool. In particular, the invention relates to a multi-functional surgical tool that has a tissue separation means, a cutting means, and a grasping means, with a buffer region between the cutting and grasping means. The invention particularly relates to a multi-functional surgical tool that is suitable for use in endoscopic or laparoscopic surgery, but may also be used in open surgery.

### BACKGROUND OF THE INVENTION

Surgery typically involves the dissection of one or more layers of tissue in order to gain access to damaged or diseased tissue for repair or removal. Sharp dissection (which involves cutting tissues) is typically performed using a surgical scalpel and/or surgical scissors. There is a variety of scalpels and scissors available for use in surgery. Each different scalpel and scissors is adapted to perform a particular sharp dissection function.

Sharp dissection is usually accompanied by bleeding from dissected tissues. Bleeding during surgery must be controlled to prevent blood from obscuring tissues in the operating field. Forceps are used to grasp and occlude blood vessels in order to control bleeding. Multiple forceps can be used to clamp blood vessels during a particular surgical procedure. Alternatively, diathermy can be applied through a single pair of forceps to sequentially grasp, occlude, and cauterise multiple blood vessels. There are several different types of forceps available, each designed for a particular grasping function.

At the completion of a surgical procedure, dissected tissues are frequently sutured back together using a surgical needle and thread, needle holders, and needle receivers. Needle holders are used to advance a surgical needle and thread through dissected tissues. A needle receiver is required to receive the needle after it has been substantially advanced through tissue and to pull the needle and thread through the tissue in order to draw dissected tissues together. The needle holder and needle receiver can be identical instruments. However, a needle holder must be able to firmly grasp the needle in order to force it through tissues. The grasping means of the needle receiver need not be so powerful while the needle receiver must be able to manipulate a needle after it has been substantially passed through tissue by the needle holder and to pull the needle and thread through tissue by grasping either the needle or the thread. This function does not require the firm grasp that is required of the needle holder and may be performed by a different grasping instrument. The thread is then knotted and the suture is cut, normally by scissors.

A typical surgical procedure will therefore require at least the use of one scalpel, scissors, forceps, and needle holder/receiver. Most surgical procedures will require the use of more than one type of each of these tools in addition to a number of other surgical instruments. The need to use several different surgical tools during surgery requires the surgeon to alternate between tools and/or use several different tools simultaneously. The need to alternate between tools can increase the time taken to complete the surgery. Alternating between tools also increases the risk of unintended damage to tissues. Also, the simultaneous use of several surgical tools in the operating field can obstruct the surgeon's vision and his or her ability to appropriately manipulate tissues. The need to use more than one tool simultaneously during surgery increases the technical difficulty of the surgery placing an increased burden on the surgeon and operating room personnel.

These problems are heightened in the case of endoscopic and laparoscopic surgery, where surgery is performed inside the body of a patient using long, thin surgical tools that are inserted into the body through small (generally 5-10 mm) incisions. The operative field is visualized by the surgeon using an endoscope or laparoscope which is also inserted into the body through a small incision.

Bleeding is a significant problem in endoscopic and laparoscopic surgery. Grasping forceps cannot be used to clamp or grasp blood vessels in the same way that they are used to control bleeding in conventional open surgery. This increases the risk of blood obscuring tissues in the operative field. Visualisation of the operative field is limited to video feedback from the endoscope or laparoscope. Blood on the endoscope or laparoscope lens can obliterate vision of the operative field, and the endoscope or laparoscope must be removed, cleaned, and reinserted into the body. In addition, due to restricted access to the operating field, alternating between surgical tools during endoscopic or laparoscopic surgery is more time consuming and involves greater risk of unintended tissue damage, which in turn increases the risk of bleeding.

In an attempt to minimise the need to use several surgical tools in one surgical procedure, some surgical tools have been designed to perform more than one surgical function.

For example, U.S. Pat. No. 6,206,877 describes a surgical instrument that combines a scissor functionality with a forceps functionality. This instrument has bipolar jaw members connected by a pivoting pin. The opposing edges of the jaw members each have a flat grasping surface and a sharp cutting surface. One instrument is described where the distal portion of the edge of each jaw member is a flat surface, which provides the instrument with a grasping means, whereas the proximal portion of the edge of each jaw member forms a blade, thereby providing a cutting scissor means. Another instrument is described in which the grasping portion of the instrument is proximal and the cutting portion is distal. This instrument can be used in conjunction with diathermy to cauterise blood vessels grasped by the instrument.

The instrument of U.S. Pat. No. 6,206,877 is therefore able to dissect tissues using the scissor functionality and to grasp and cauterize blood vessels to control bleeding. However, the straight and untapered form of the jaws of this instrument limits visibility of the distal tip of the instrument when in use and restricts the ability of the instrument to access recessed structures. In addition, since the cutting means is immediately adjacent to the grasping means, there is significant risk of accidentally cutting tissues or sutures while grasping.

Furthermore, both the cutting and grasping means of the instrument are effected by closing the jaws of the instrument. No additional functionality is provided by the opening action of the jaws. The leading edge of the instrument is untapered and blunt. Thus, the closed jaws of the instrument can not effectively be inserted between adjacent tissues to enable separation of tissue when the jaws of the instrument are opened.

U.S. Pat. No. 6,790,217 describes a surgical instrument designed for use in endoscopic or laparoscopic surgery. This instrument has a means for cutting, grasping, and cauterising tissue. The cutting means of this instrument is complex compared to the simple dual-function jaw design of U.S. Pat. No. 6,206,877. The cutting means described in U.S. Pat. No. 6,790,217 comprises a cutting element positioned in a channel that runs through the grasping jaws of the instrument. In use, tissue is grasped by the jaws and cauterised while the cutting element is in an extended position. Upon appropriate manipulation of the instrument by the surgeon, the cutting element is retracted into the shaft of the instrument allowing a first blade on the cutting element to transect tissues that are grasped between the jaws of the instrument. The cutting element also has a sharpened distal tip that is able to transect grasped tissues when the cutting element is allowed to move from its retracted position back through the channel in the jaw members.

Retracting the cutting element of the instrument reduces the risk of accidentally cutting tissues. However, alternating the cutting element between the retracted position and the extended position can be cumbersome in laparoscopic adhesiolysis and surgeons will frequently find it easier to keep the cutting blade in the extended position. Thus, in practice, the advantage provided by the retractable blade is defeated. In addition, U.S. Pat. No. 6,790,217 does not describe an instrument that has an effective means to enable the separation of tissues or which has a needle holding or receiving functionality.

The instrument described in U.S. Pat. No. 6,514,267 is an example of a harmonic scalpel. A harmonic scalpel has the ability to grasp, cauterise, and then cut tissues. However, the use of the harmonic scalpel is expensive and slow. Cauterisation prior to cutting causes unnecessary damage to tissues and causes the formation of adhesions. Prior to grasping any bleeding blood vessels, the surgeon must wait for the jaws to cool sufficiently to avoid burning any grasped tissue. There is also the danger of burning due to accidental contact of the jaws with tissue. In addition, the harmonic scalpel does not have the functionality of a pair of surgical scissors.

U.S. Pat. No. 6,773,434 describes a surgical instrument designed for use in endoscopic or laparoscopic surgery. This instrument employs various means for cutting, grasping, and cauterising tissue. The document also describes an instrument having curved jaws to provide improved visibility of the distal tip of the instrument when in use and improved access to recessed structures. Also described is an instrument having tapered jaws and an instrument having a sharpened distal tip for transecting tissues.

U.S. Pat. No. 6,773,434 describes a single instrument claimed to have tissue cutting, tissue grasping, and tissue separating functions. However, there is description of an instrument that has an effective means to enable the separation of tissues or which has a needle holding or receiving functionality. This instrument also suffers from the same disadvantages as the instrument of U.S. Pat. No. 6,790,217, described above.

The applicant has devised a simple multifunctional surgical tool that provides additional functionality over known surgical tools and avoids many of the limitations of known multifunctional surgical tools. The tool of the invention is able to control bleeding during surgery and to reduce the problems associated with alternating between surgical tools and with using several surgical tools simultaneously.

### SUMMARY OF THE INVENTION

It is therefore an object of this invention to provide a simple multi-functional surgical tool that is able to overcome at least some of the limitations of known surgical tools, or to at least provide a useful choice.

In a first aspect of the invention there is provided a surgical tool having:

a) first and second jaw members pivotally connected to allow the jaw members to move between an open position and a closed position;

b) a grasping means comprising a grasping surface on the first jaw member and an opposed grasping surface on the second jaw member enabling grasping when the jaw members close;

c) a cutting means comprising a cutting portion on the first jaw member and an opposed cutting portion on the second jaw member enabling cutting when the jaw members close; and

d) a buffer region between the grasping surface and the cutting portion of the first jaw member and an opposed buffer region between the grasping surface and the cutting portion of the second jaw member.

In a preferred embodiment of the invention the distal end of the tool, when the jaw members are in the closed position, is sufficiently narrow to allow the tool to be inserted between adjacent tissues thereby allowing the tissues to be separated when the jaw members are opened.

Preferably the buffer region has a smooth convex surface.

Preferably the cutting portions are positioned at the proximal end of each jaw member and the grasping surfaces are positioned at the distal end of each jaw member.

Preferably the dimensions of the cutting portion, grasping surface, and buffer region on one jaw member are the essentially the same as the dimensions of the equivalent parts on the other jaw member.

Preferably the width of each jaw member at the widest point of the grasping surface is at least twice the width of each jaw member at the cutting portion.

Preferably the surface area of each grasping surface is sufficient to effectively grasp and hold tissue, suture thread, or a needle.

The grasping surface of each jaw member may be serrated (preferable for holding tissue) or may be non-serrated (preferable for holding suture thread or needles).

Preferably each jaw member is tapered so that it narrows to a point.

Preferably each jaw member is curved to provide improved visibility of the distal end of the tool when in use and improved access to recessed structures when the tool is in use.

The tool may be adapted for use with diathermy.

Preferably the surface area of each grasping surface is sufficient to cauterise grasped tissue. Cauterisation of grasped tissue is preferably achieved using an offset electrode arrangement.

In one embodiment of the invention, the tool is adapted for use in endoscopic or laparoscopic surgery. Preferably the tool has dimensions which enable it to be inserted through 5 to 10 mm entry incisions in the body of a patient. The tool is preferably adapted for use with a variety of known endoscopic and laparoscopic tools.

In an alternative embodiment of the invention, the tool is adapted for use in open surgery. Preferably the tool is adapted for use with a variety of known open surgery tools.

In a second aspect of the invention there is provided a use of the surgical tool of the first aspect of this invention in a surgical procedure.

The surgical procedure is preferably an endoscopic or laparoscopic surgical procedure, but may alternatively be an open surgical procedure.

Preferably, the use of the tool in a surgical procedure includes:

a) cutting tissue with the cutting means; and

b) grasping tissue with the grasping means.

It is also preferred that the use of the tool in a surgical procedure includes:

c) inserting the distal end of the tool between adjacent tissues when the jaw members are closed; and

d) separating tissues by opening the jaw members.

It is further preferred that the use of the tool in a surgical procedure includes:

e) grasping and manipulating a surgical needle with the grasping means;

f) grasping and manipulating surgical thread with the grasping means; and

g) cutting surgical thread with the cutting means.

The use of the tool in a surgical procedure may also include cauterising tissue that is grasped by the grasping means.

### BRIEF DESCRIPTION OF THE DRAWINGS

FIG. 1 is a perspective view of a preferred embodiment of the tool of the invention when the jaw members of the tool are in the open position.

FIG. 2 is another perspective view of the tool illustrated in FIG. 1 when the jaw members are in the open position.

FIG. 3 is perspective view of the tool illustrated in FIG. 1 when the jaw members are in the closed position.

FIG. 4 is a perspective view of the second jaw member of the tool illustrated in FIG. 1; and,

FIG. 5 is a perspective view of the first jaw member of the tool illustrated in FIG. 1.

### DETAILED DESCRIPTION OF THE INVENTION

The invention provides a simple multi-functional surgical tool that has a cutting means for cutting tissue and surgical thread, and a means to grasp and hold tissues and surgical needles. Importantly, the tool has a buffer region between the cutting and grasping means. Optionally, the tool also has a means to cauterise tissue. The tool may be adapted for use in endoscopic or laparoscopic surgery or in open surgery. The tool preferably also has a means for separating tissue.

The multi-functionality of the tool reduces the need to alternate between tools and the need to use several different tools simultaneously. The cutting means provides for effective dissection of tissues and the cutting of surgical thread. The grasping and optional cauterising means of the invention provide for effective control of bleeding. The grasping means also provides an effective needle holding or receiving means. The tissue separation means enables the separation of adjacent tissues.

The invention provides a surgical tool having first and second jaw members. The second jaw member is pivotally coupled to the first jaw member so that the jaw members are pivotable between an open position and a closed position. When the tool is being closed, either jaw member or both jaw members can be pivotably rotated so that one jaw member is gradually brought into contact with the other jaw member.

The first and second jaw members each have a cutting portion (the first cutting portion and the second cutting portion, respectively). In the open position, the first and second cutting portions are in contact only at the proximal end of the tool relative to the pivot. When the tool is being closed, the cutting portions are gradually brought into contact with each other along the entire length of the cutting portions. At least one of the first and second cutting portions has a sharpened leading edge capable of cutting tissue that is positioned between the first and second jaw members.

In addition, the first and second jaw members each have a grasping surface (the first grasping surface and the second grasping surface, respectively). The grasping surfaces are preferably at the distal end of the jaw members. The first grasping surface is positioned opposite the second grasping surface when the jaws of the tool are open. When the jaws of the tool are closed, the first and second grasping surfaces are in close contact.

The jaw members are therefore capable of moving between an open and a closed position such that the first and second cutting portions form a scissor means and the grasping surfaces form a grasping means. A surgeon can utilise the scissor means for dissection and, should he or she encounter bleeding, can grasp the bleeding blood vessel with the grasping means to control bleeding.

The first cutting portion of the tool is separated from the first grasping surface on the first jaw member by a first buffer region. Similarly, the second cutting portion is separated from the second grasping surface on the second jaw member by a second buffer region. The first and second buffer regions have neither a grasping nor a cutting functionality. They serve to create space between the scissor means and the grasping means to reduce the risk of accidentally cutting tissues whilst grasping. The buffer regions can therefore have a range of dimensions. Preferably the buffer region has a smooth convex surface.

The tool of the invention can be used in conjunction with diathermy whereby the surgeon can cauterise grasped tissues before cutting the tissues. This greatly reduces the area of tissue coagulated and significantly reduces the risk of unintended tissue damage. Preferably, cauterisation of grasped tissue is achieved using an offset electrode arrangement of the kind described in U.S. Pat. No. 6,773,434 and EP 0722696.

The grasping means of the tool can be used as a needle holder or receiver during suturing. Suturing tissues involves a needle holder which is used to advance a surgical needle and thread through dissected tissues in order to draw the tissues together. A needle receiver is required to receive a needle which has been substantially advanced through tissue and to pull the needle and thread through the tissue. The thread is then knotted and the suture is cut.

The grasping means of the tool can be used as a needle holder or receiver. The grasping surfaces may be serrated or non-serrated to improve the tool's grip on the needle, as required. The cutting means of the tool can be used to cut sutures. Use of the tool therefore reduces the need to use separate needle receivers and scissors.

The tool additionally has a tissue separation means. Typically, the first and second jaw members are both tapered such that each jaw member narrows along its length to a point at its distal end. The point may be blunt or sharpened.

When the tool is closed, its end is sufficiently narrow to be inserted between adjacent tissues. The tissues can then be separated by opening the jaws of the tool. Adjacent tissues can be separated intact. For example, a blood vessel can be separated from fatty or connective tissue without rupturing the blood vessel.

The dimensions of the first jaw member are typically the same as the dimensions of the second jaw member. It is also preferred that the dimensions of the cutting portion, grasping surface, and buffer region of the first jaw member are the same as the dimensions of the equivalent parts on the second jaw member. For clarity, the following description of the invention assumes this preferred feature. However, the invention contemplates that the dimensions of the first and second jaw members or their cutting portions, grasping surfaces, and buffer regions need not be identical for the object of the invention to be achieved. A person skilled in the art will appreciate that the dimensions of the tool, and of the cutting portions, grasping surfaces, and buffer regions, can vary according to the different surgical purposes to which the invention may be adapted.

The width of each jaw member is generally constant along the length of the cutting portion and buffer region.

Typically the width of each jaw member at the grasping surface is larger than the width of the jaw member along the length of the cutting portion and buffering region. The width of each jaw member at its grasping surface can be variable. The width of each jaw member at the widest point of the grasping surface is typically at least twice the width of each jaw member along the length of the cutting portion and buffering region. The surface area of each grasping surface is therefore sufficient to effectively grasp and hold tissues.

When the grasping surfaces are positioned at the distal end of the jaw members, the distal end of the tool when closed is narrow enough to allow the tool to be inserted between adjacent tissues so that the grasping surfaces of the tool are substantially inserted between the tissues and the outer surface of each jaw member that is opposite the grasping region is in contact with tissue.

Typically the outer surface of each jaw member has the same surface area as the grasping region. The surface area of the outer surface of each jaw member is sufficient to prevent shearing of tissues when tissues are separated as the jaw members are opened.

The tool of the invention is preferably curved to provide improved visibility of the distal end of the instrument and improved access to recessed structures, when in use.

The tool of the invention can be adapted for use in endoscopic or laparoscopic surgery, or for use in open surgery. In particular, the tool of the invention can have dimensions which enable it to be inserted through the small (generally 5 to 10 mm) entry incisions used in endoscopic or laparoscopic surgery. The tool can be adapted for use with a variety of known endoscopic and laparoscopic tools.

The tool of the invention can be constructed from any suitable material.

The invention also provides a use of the tool of the invention in a surgical procedure. The surgical procedure may involve inserting the distal end of the tool when the jaw members are closed between adjacent tissues; separating tissues by opening the jaw members; cutting tissue with the cutting means; grasping tissue with the grasping means; grasping and manipulating a surgical needle with the grasping means; grasping and manipulating surgical thread with the grasping means; and cutting surgical thread with the cutting means. Typically the surgical procedure will also involve cauterising tissue that is grasped by the grasping means. An example is laparoscopic surgery for adhesiolysis.

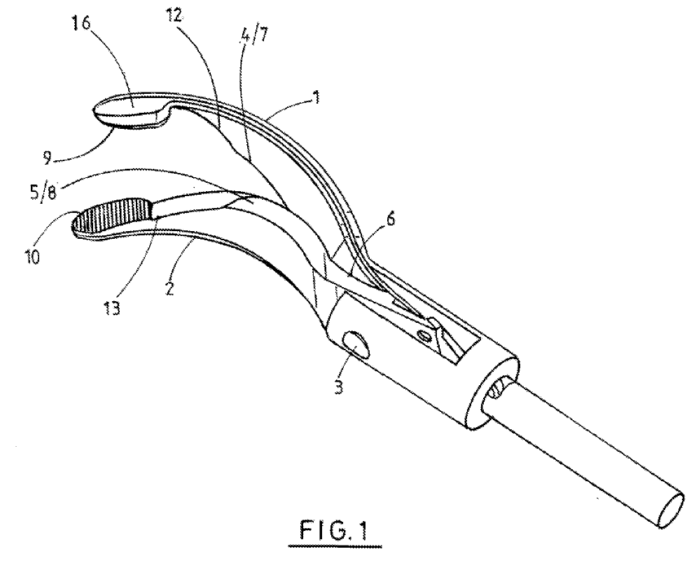

The invention is further described with reference to the embodiment shown in FIGS. 1 to 5. However, it is to be appreciated that the invention is not limited to this embodiment.

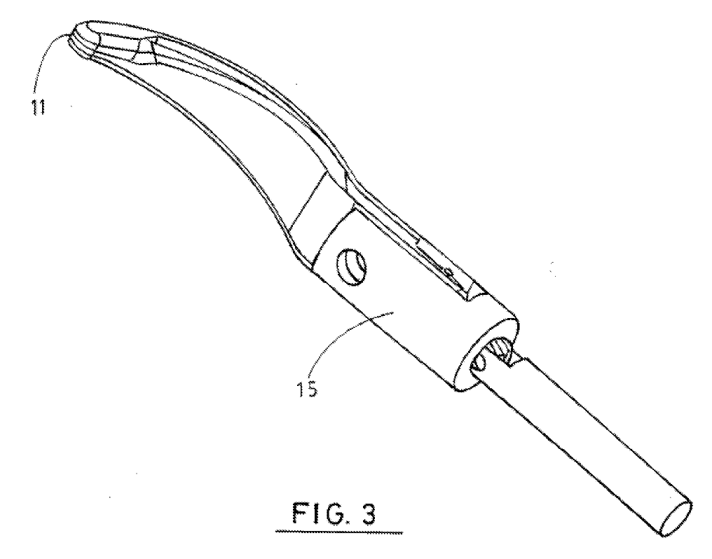

FIG. 1 illustrates a preferred embodiment of the tool of the invention when the jaw members are in the open position. The tool has a first jaw member (1) and a second jaw member (2). The second jaw member (2) is coupled to the first jaw member (1) by a pivot (3). The second jaw member (2) is therefore pivotable relative to the first jaw member (1) between the open position and a closed position (shown in FIG. 3).

When the jaws are moved from an open position to a closed position, one jaw member may remain static while the other jaw member is moved toward it, or alternatively both jaw members may move simultaneously toward each other.

The first and second jaw members (1 and 2) each have a cutting portion at the proximal end of each jaw member relative to the pivot (the first cutting portion (4) and the second cutting portion (5), respectively). When the jaw members (1 and 2) are in the open position, the first and second cutting portions (4 and 5) are in contact only at the proximal pivot end of the tool (6). When the tool is being closed, the second cutting portion (5) is gradually brought into contact with the first cutting portion (4) along the entire length of the second cutting portion (5). The second cutting portions (4) is shown having a sharpened leading edge (8).

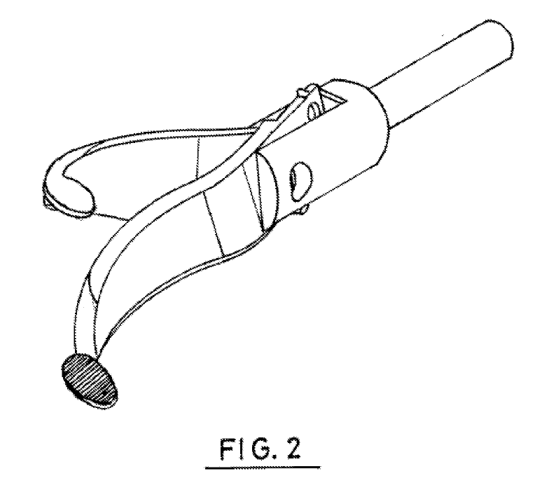

The jaw members (1 and 2) each have a grasping surface, the first grasping surface (9) and the second grasping surface (10). The grasping surfaces (9 and 10) are at the distal end of the tool. The first grasping surface (9) is positioned opposite the second grasping surface (10) when the jaws of the tool are open (illustrated in FIGS. 1 and 2). When the jaws of the tool are closed, the first and second grasping surfaces (9 and 10) meet and are in close contact (see (11) in FIG. 3).

FIG. 1 also illustrates that the first cutting portion (4) is separated from the first grasping surface (9) on the first jaw member (1) by a first buffer region (12). Similarly, the second cutting portion (5) is separated from the second grasping surface (10) on the second jaw member (2) by a second buffer region (13). The buffer regions (12 and 13) have neither a grasping nor a cutting functionality. They serve to create space between the cutting means and the grasping means to reduce the risk of accidentally cutting tissues while grasping. The buffer regions (12 and 13) have a smooth convex surface.

The dimensions of the cutting portions (4 and 5), grasping surfaces (9 and 10), and buffer regions (12 and 13) of the first jaw member (1) are essentially the same as the dimensions of the equivalent parts on the second jaw member (2).

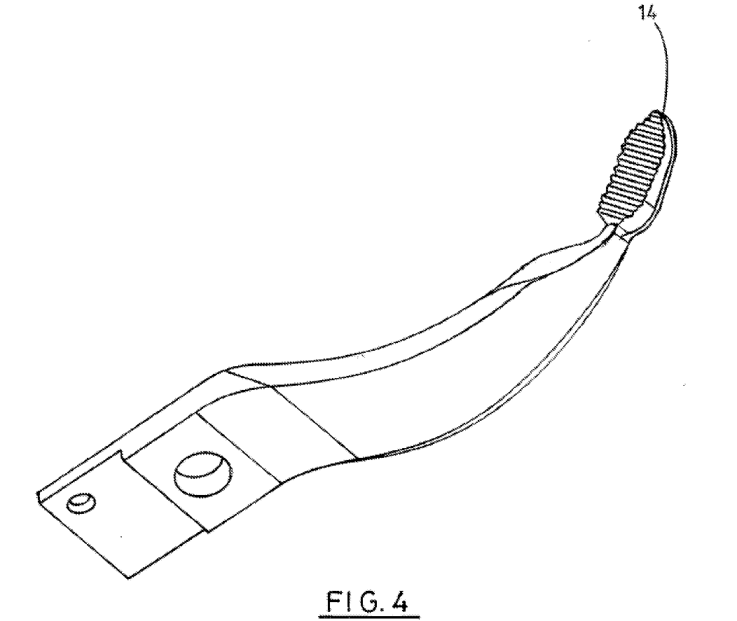

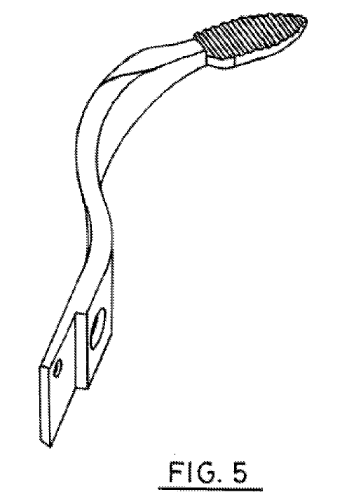

The width of each jaw member (1 and 2) is constant along the length of the cutting portions (4 and 5) and buffer regions (12 and 13) (this is illustrated for each jaw member in FIGS. 4 and 5). The width of each jaw member (1 and 2) at the grasping surfaces (9 and 10) is larger than the width of each jaw member (1 and 2) along the length of the cutting portions (4 and 5) and buffer regions (12 and 13). The width of each jaw member (1 and 2) at the widest point of the grasping surface (9 and 10) is about twice the width of each jaw member (1 and 2) along the length of each cutting portion (4 and 5) and each buffer region (12 and 13).

Each jaw member (1 and 2) is tapered so that it narrows to a point at the distal end of each jaw member (see (14) in FIG. 4). The point of each jaw member (1 and 2) is blunt. Thus, when the jaw members are closed, the height of the distal end of the tool ((11), illustrated in FIG. 3) is less than the height of the proximal end of the tool (15). The distal end of the tool, when in the closed position, is sufficiently narrow to allow it to be inserted between adjacent tissues, and the tissues then separated when the jaw members (1 and 2) are opened.

The surface area of the outer surface (16) of each jaw member (1 and 2) is sufficient to prevent shearing of tissues when tissues are separated as the jaw members (1 and 2) are opened.

The tool is curved to provide improved visibility of the distal end and improved access to recessed structures, when in use.

The tool is adapted for use in endoscopic or laparoscopic surgery. In particular, the tool is able to be inserted through a 5 to 10 mm entry incision in the body of a patient. The tool is also adapted for use with a variety of known endoscopic and laparoscopic tools.

Although the invention has been described by way of example, it should be appreciated that variations and modifications may be made without departing from the scope of the claims. Furthermore, where known equivalents exist to specific features, such equivalents are incorporated as if specifically referred to in this specification.

The invention provides a simple multi-functional surgical tool that has a cutting means for cutting tissue and surgical thread, and a means to grasp and hold tissues and surgical needles. Optionally, the surgical tool of the invention also has a means to cauterise tissue. The tool may be used in endoscopic or laparoscopic surgery or in open surgery. The tool preferably also has a means for separating tissue. The multi-functionality of the tool reduces the need to alternate between tools and the need to use several different tools simultaneously. The cutting means provides for effective dissection of tissues and the cutting of surgical thread. The grasping and optional cauterising means of the invention provide for effective control of bleeding. The grasping means also provides an effective needle holding or receiving means. The tissue separation means enables the separation of adjacent tissues.

1. A surgical tool having:
a) first and second jaw members pivotally connected to allow the jaw members to move between an open position and a closed position;
b) a grasping means comprising a grasping surface on the first jaw member and an opposed grasping surface on the second jaw member enabling grasping when the jaw members close;
c) a cutting means comprising a cutting portion on the first jaw member and an opposed cutting portion on the second jaw member enabling cutting when the jaw members close; and
d) a buffer region between the grasping surface and the cutting portion of the first jaw member and an opposed buffer region between the grasping surface and the cutting portion of the second jaw member.

2. A surgical tool as claimed in claim 1 where the distal end of the tool, when the jaw members are in the closed position, is sufficiently narrow to allow the tool to be inserted between adjacent tissues thereby allowing the tissues to be separated when the jaw members are opened.

3. A surgical tool as claimed in claim 1 where the buffer region has a smooth convex surface.

4. A surgical tool as claimed in claim 1 where the cutting portions are positioned at the proximal end of each jaw member and the grasping surfaces are positioned at the distal end of each jaw member.

5. A surgical tool as claimed in claim 1 where the dimensions of the cutting portion, grasping surface, and buffer region on one jaw member are the essentially the same as the dimensions of the equivalent parts on the other jaw member.

6. A surgical tool as claimed in claim 1 where the width of each jaw member at the widest point of the grasping surface is at least twice the width of each jaw member at the cutting portion.

7. A surgical tool as claimed in claim 1 where the surface area of each grasping surface is sufficient to grasp and hold tissue, suture thread, or a needle.

8. A surgical tool as claimed in claim 1 where the grasping surface of each jaw member is serrated.

9. A surgical tool as claimed in claim 1 where the grasping surface of each jaw member is non-serrated.

10. A surgical tool as claimed in claim 1 where each jaw member is tapered so that it narrows to a point.

11. A surgical tool as claimed in claim 1 where each jaw member is curved to provide improved visibility of the distal end of the tool when in use and improved access to recessed structures when the tool is in use.

12. A surgical tool as claimed in claim 1 where the tool is adapted for use with diathermy.

13. A surgical tool as claimed in claim 1 where the surface area of each grasping surface is sufficient to cauterise grasped tissue.

14. A surgical tool as claimed in claim 1 where an offset electrode arrangement is used to cauterise grasped tissue.

15. A surgical tool as claimed in claim 1 which is adapted for use in endoscopic or laparoscopic surgery.

16. A surgical tool as claimed in claim 15 having dimensions which enable it to be inserted through 5 to 10 mm entry incisions in the body of a patient.

17. A surgical tool as claimed in claim 15 which is adapted for use with another endoscopic or laparoscopic tool.

18. A surgical tool as claimed in claim 1 which is adapted for use in open surgery.

19. A surgical tool as claimed in claim 18 which is adapted for use with another surgical tool.

20. The use of the surgical tool of claim 1 in a surgical procedure.

21. The use as claimed in claim 20 where the surgical procedure is an endoscopic or laparoscopic surgical procedure.

22. The use as claimed in claim 21 where the surgical procedure is an open surgical procedure.

23. The use as claimed in claim 20 where the surgical procedure includes:
a) cutting tissue with the cutting means; and
b) grasping tissue with the grasping means.

24. The use as claimed in claim 20 where the surgical procedure includes:
a) inserting the distal end of the tool between adjacent tissues when the jaw members are closed; and
b) separating the tissues by opening the jaw members.

25. The use as claimed in claim 20 where the surgical procedure includes:
a) grasping and manipulating a surgical needle with the grasping means;
b) grasping and manipulating surgical thread with the grasping means; and
c) cutting surgical thread with the cutting means.

26. The use as claimed in claim 20 where the surgical procedure includes cauterising tissue that is grasped by the grasping means.
